(19) United States
(12) Patent Application Publication
(10) Pub. No.: US 2025/0143817 A1
(43) Pub. Date: May 8, 2025

(54) AUTOMATIC ADAPTIVE MODE FOR SURGICAL ROBOTIC SYSTEM

(71) Applicants: Covidien LP, Mansfield, MA (US); Digital Surgery Limited, London (GB)

(72) Inventors: Jullian C. Cockerell, Minneapolis, MN (US); Gary J. Suszynski, Woodbury, CT (US); Christopher P. Penna, Guilford, CT (US); Kevin S. Sniffin, Roxbury, CT (US); Henry E. Holsten, Sumter, SC (US); Jonathan D. Thomas, New Haven, CT (US); Danail V. Stoyanov, London (GB)

(21) Appl. No.: 18/867,869

(22) PCT Filed: Jun. 8, 2023
(86) PCT No.: PCT/EP2023/065429

§ 371 Date: Nov. 21, 2024

Related U.S. Application Data
(60) Provisional application No. 63/462,577, filed on Apr. 28, 2023.
(60) Provisional application No. 63/355,179, filed on Jun. 24, 2022.
(60) Provisional application No. 63/355,183, filed on Jun. 24, 2022.
(60) Provisional application No. 63/355,191, filed on Jun. 24, 2022.

(51) Int. Cl. A61B 34/32; A61B 34/00; A61B 34/35

(52) U.S. Cl. CPC A61B 34/32; A61B 34/35; A61B 34/74

(57) ABSTRACT

A surgical robotic system includes a robotic arm having an instrument and an instrument drive unit configured to couple to and to actuate the instrument. The system also includes a surgeon console having a handle controller configured to control the robotic arm and the instrument. The system further includes a processor configured to monitor a parameter of the instrument and select a power mode from a plurality of power modes for the instrument drive unit based on the parameter. The plurality of power modes includes a full power mode during which the instrument drive unit is fully powered and a low power mode during which the instrument drive unit is partially powered. The processor is further configured to set the instrument drive unit to the selected power mode.

### CROSS REFERENCE TO RELATED APPLICATIONS

This application claims the benefit of and priority to U.S. Provisional Patent Application Ser. No. 63/355,179 filed on Jun. 24, 2022; U.S. Provisional Patent Application Ser. No. 63/355,183 filed on Jun. 24, 2022; U.S. Provisional Patent Application No. 63/355,191, filed on Jun. 24, 2022; and U.S. Provisional Patent Application No. 63/462,577 filed on Apr. 28, 2023. The entire disclosures of the foregoing applications are incorporated by reference herein.

### BACKGROUND

Surgical robotic systems generally include a surgeon console controlling one or more surgical robotic arms, each including a surgical instrument having an end effector (e.g., forceps or grasping instrument). In operation, the robotic arm is moved to a position over a patient and the surgical instrument is guided into a small incision via a surgical access port or a natural orifice of a patient to position the end effector at a work site within the patient's body. The surgeon console includes hand controllers which translate user input into movement of the surgical instrument and/or end effector. During use, various components of the robotic arm and the surgical instrument experience significant wear and tear due to repeated actuation of motors, drive trains, cables, pulleys, etc. Thus, there is a need for a system and method to maintain responsiveness while minimizing wear on the components.

### SUMMARY

The present disclosure provides for a surgical robotic system that may be operated in an adaptive manner to reduce wear and tear of surgical instruments and other components as well as provide more responsive control of the instrument. The surgical robotic system includes a surgical instrument that is held by a robotic arm. The robotic arm includes an instrument drive unit (IDU) having one or more motors that are configured to actuate (i.e., adjust yaw and/or pitch, pivot, open/close jaws, engage a knife, etc.) the surgical instrument. The surgical robotic system also includes a surgeon console including one or more handle controllers configured to receive user input and actuate the surgical instrument in response thereto. The adaptive operation allows the system to switch between two or more operation modes, i.e., full power and low power modes. During full power (i.e., active) mode the IDU is activating the motors to apply full mechanical power that the instrument is capable of. During the low power mode, the system is configured to minimize the mechanical power imparted by the instrument. This may include operating the IDU at a lower current, lower torque thresholds, and the like. The provided power modes are exemplary and any desired varying power or torque modes are contemplated.

Mode switching may be done either manually, i.e., by a user, or automatically, i.e., by the system in response to variety of events, such as, based on a phase of surgery, monitoring user engagement with the surgeon console, usage of the instrument, etc. In embodiments, the lower power mode may include one or more levels with varying degrees of reduction in mechanical power of the surgical instrument. Operation of the surgical instrument in a continuous full power mode dramatically shortens useful life due to the stresses imparted on the mechanical components. Thus, reducing the amount of time that the instruments are operating in the full power mode, and are instead operating in a lower power mode, will increase instrument life.

According to one embodiment of the present disclosure, a surgical robotic system is disclosed. The surgical robotic system includes a robotic arm having an instrument and an instrument drive unit configured to couple to and to actuate the instrument. The system also includes a surgeon console having a handle controller configured to control the robotic arm and the instrument. The system further includes a processor configured to monitor a parameter of the instrument and select a power mode from a plurality of power modes for the instrument drive unit based on the parameter. The plurality of power modes includes a full power mode during which the instrument drive unit is fully powered and a low power mode during which the instrument drive unit is partially powered. The processor is further configured to set the instrument drive unit to the selected power mode.

Implementations of the above embodiment may include one or more of the following features. According to one aspect of the above embodiment, the processor may be further configured to determine whether the handle controller is clutched and to select the power mode from the plurality of power modes based on a clutch status. The processor may be further configured to select the low power mode when the handle controller is clutched out. The processor may be also configured to select the full power mode when the handle controller is clutched in. The processor may be further configured to determine whether the instrument is inside or outside a patient. The processor may be further configured to select the low power mode when the instrument is disposed outside the patient. The processor may be additionally configured to select the full power mode when the instrument is disposed inside the patient.

According to another embodiment of the present disclosure, a surgical robotic system is disclosed. The surgical robotic system includes a robotic arm having an instrument and an instrument drive unit configured to couple to and to actuate the instrument. The system also includes a processor configured to monitor a parameter of the instrument and select a power mode from a plurality of power modes for the instrument drive unit based on the parameter. The plurality of power modes includes a full power mode during which the instrument drive unit is fully powered and a low power mode during which the instrument drive unit is partially powered. The processor is further configured to set the instrument drive unit to the selected power mode.

Implementations of the above embodiment may include one or more of the following features. According to one aspect of the above embodiment, the processor may be further configured to determine whether the instrument is inside or outside a patient. The processor may be also configured to select the low power mode when the instrument is disposed outside the patient. The processor may be also configured to select the full power mode when the instrument is disposed inside the patient. The processor may be additionally configured to determine whether the instrument is idle or is in use. The processor may be configured to select the low power mode when the instrument is idle. The processor may be also configured to select the full power mode when the instrument is in use. The processor may be further configured to determine whether the instrument is grasping tissue. The processor may be further configured to select the low power mode when the instrument is not grasping tissue. The processor may be also configured to select the full power mode when the instrument is grasping tissue.

According to a further embodiment of the present disclosure, a method for controlling a surgical robotic system is disclosed. The method includes monitoring a parameter of an instrument coupled to an instrument drive unit disposed on a robotic arm. The instrument drive unit is configured to couple to and to actuate the instrument. The method also includes monitoring input at a surgeon console having a handle controller configured to control the robotic arm and the instrument. The method further includes selecting a power mode from a plurality of power modes for the instrument drive unit based on the parameter. The plurality of power modes includes a full power mode during which the instrument drive unit is fully powered and a low power mode during which the instrument drive unit is partially powered. The method additionally includes setting the instrument drive unit to the selected power mode.

Implementations of the above embodiment may include one or more of the following features. According to one aspect of the above embodiment, the method may also include: determining at a processor whether the instrument is idle or is in use; selecting the low power mode when the instrument is idle; and selecting the full power mode when the instrument is in use. The method may further include: determining at a processor whether the instrument is grasping tissue; selecting the low power mode when the instrument is not grasping tissue; and selecting the full power mode when the instrument is grasping tissue.

According to one embodiment of the present disclosure, a surgical robotic system is disclosed. The surgical robotic system includes a robotic arm having an instrument and an instrument drive unit configured to couple to and to actuate the instrument. The system also includes a display configured to output a graphical user interface and a surgeon console having a handle controller configured to control the robotic arm and the instrument. The system further includes a processor configured to receive a user input from the graphical user interface, the user input selecting a power mode from a plurality of power modes for the instrument drive unit. The plurality of power modes includes a full power mode during which the instrument drive unit is fully powered and a low power mode during which the instrument drive unit is partially powered. The processor is further configured to set the instrument drive unit to the selected power mode.

Implementations of the above embodiment may include one or more of the following features. According to one aspect of the above embodiment, the surgical robotic system may also include a storage device storing user settings having a preset power mode. The processor may be further configured to receive the user settings and the set the instrument drive unit to the preset power mode. The graphical user interface may be configured to display the plurality of power modes. The display may be a touchscreen and the user input may include touching the power mode. The handle controller may include an input device configured to select the power mode. The input device may be configured to measure a grip force of the handle controller. The processor may be further configured to select the power mode based on the grip force.

According to another embodiment of the present disclosure, a surgical robotic system is disclosed. The surgical robotic system may include a robotic arm that includes an instrument and an instrument drive unit configured to couple to and to actuate the instrument. The system also includes a display configured to output a graphical user interface. The system further includes a processor configured to receive a user input from the graphical user interface, the user input selecting a power mode from a plurality of power modes for the instrument drive unit. The plurality of power modes includes a full power mode during which the instrument drive unit is fully powered and a low power mode during which the instrument drive unit is partially powered. The processor is further configured to set the instrument drive unit to the selected power mode.

Implementations of the above embodiment may include one or more of the following features. According to one aspect of the above embodiment, the surgical robotic system may include a storage device storing user settings having a preset power mode. The processor is further configured to receive the user settings and the set the instrument drive unit to the preset power mode. The graphical user interface may be also configured to display the plurality of power modes. The display may be a touchscreen and the user input may include touching the power mode. The handle controller may also include an input device configured to select the power mode. The input device may be configured to measure a grip force of the handle controller. The processor may be also configured to select the power mode based on the grip force.

According to a further embodiment of the present disclosure, a method for controlling a surgical robotic system is disclosed. The method includes receiving an input at a handle controller for moving an instrument coupled to an instrument drive unit disposed on a robotic arm. The instrument drive unit is configured to couple to and to actuate the instrument. The method also includes displaying a graphical user interface on a display. The method further includes receiving a user input from the graphical user interface. The method additionally includes selecting via the user input a power mode from a plurality of power modes for the instrument drive unit. The plurality of power modes includes a full power mode during which the instrument drive unit is fully powered and a low power mode during which the instrument drive unit is partially powered. The method also includes setting the instrument drive unit to the selected power mode.

Implementations of the above embodiment may include one or more of the following features. According to one aspect of the above embodiment, displaying the graphical user interface may further include displaying the plurality of power modes. Receiving the user input may also include receiving a touch input. The method may further include measuring a grip force of the handle controller and selecting the power mode based on the grip force.

According to one embodiment of the present disclosure, a surgical robotic system is disclosed. The surgical robotic system includes a robotic arm having an instrument and an instrument drive unit configured to couple to and to actuate the instrument. The system also includes a display configured to output a graphical user interface and a surgeon console including a handle controller configured to control the robotic arm and the instrument. The system further includes a processor configured to calculate a current level of use of the instrument based on movement of the handle controller and to output on the graphical user interface an indicator showing the current level of use of the instrument.

Implementations of the above embodiment include one or more of the following features. According to one aspect of the above embodiment, the indicator may be a hysteresis gauge. The processor may be further configured to monitor a parameter of the instrument and select a power mode from a plurality of power modes for the instrument drive unit based on an instrument parameter. The plurality of power modes may include a full power mode during which the instrument drive unit is fully powered and a low power mode during which the instrument drive unit is partially powered. The processor may be further configured to set the instrument drive unit to the selected power mode. The processor may be also configured to receive a user input from the graphical user interface, the user input selecting a power mode from the plurality of power modes for the instrument drive unit. The processor may be further configured to calculate the level of use based on the selected power mode. The current level of use may be at least one of an instantaneous value or a running average value.

According to another embodiment of the present disclosure, a method for controlling a surgical robotic system is disclosed. The method includes receiving an input at a handle controller for moving an instrument coupled to an instrument drive unit disposed on a robotic arm, the instrument drive unit configured to couple to and to actuate the instrument. The method also includes displaying a graphical user interface on a display. The method further includes receiving a user input from the graphical user interface. The method also includes calculating a current level of use of the instrument based on movement of the handle controller and outputting on the graphical user interface an indicator showing the current level of use of the instrument.

Implementations of the above embodiment may include one or more of the following features. According to one aspect of the above embodiment, calculating the current level of use may include calculating at least one of an instantaneous value or a running average value. Outputting on the graphical user interface may also include displaying a hysteresis gauge. The method may further include: monitoring a parameter of the instrument and selecting a power mode from a plurality of power modes for the instrument drive unit based on an instrument parameter. The plurality of power modes may include a full power mode during which the instrument drive unit is fully powered and a low power mode during which the instrument drive unit is partially powered. The method may also include setting the instrument drive unit to the selected power mode. Calculating the current level of use is based on the selected power mode.

### BRIEF DESCRIPTION OF THE DRAWINGS

Various embodiments of the present disclosure are described herein with reference to the drawings wherein:

FIG. 1 is a schematic illustration of a surgical robotic system including a control tower, a console, and one or more surgical robotic arms each disposed on a mobile cart according to an embodiment of the present disclosure;

FIG. 2 is a perspective view of a surgical robotic arm of the surgical robotic system of FIG. 1 according to an embodiment of the present disclosure;

FIG. 3 is a perspective view of a mobile cart having a setup arm with the surgical robotic arm of the surgical robotic system of FIG. 1 according to an embodiment of the present disclosure;

FIG. 4 is a schematic diagram of a computer architecture of the surgical robotic system of FIG. 1 according to an embodiment of the present disclosure;

FIG. 5 is a perspective view, with parts separated, of an instrument drive unit and a surgical instrument according to an embodiment of the present disclosure;

FIG. 6 is a flow chart of a method for automatically adjusting a power mode of the instrument drive unit according to an embodiment of the present disclosure;

FIG. 7 is a flow chart of a method for adjusting a power mode of the instrument drive unit based on user settings according to an embodiment of the present disclosure;

FIG. 8 is a flow chart of a method for updating a level of use indicator of the instrument drive unit according to an embodiment of the present disclosure; and

FIG. 9 is an image of a display with a graphical user interface according to an embodiment of the present disclosure.

### DETAILED DESCRIPTION

Embodiments of the presently disclosed surgical robotic system are described in detail with reference to the drawings, in which like reference numerals designate identical or corresponding elements in each of the several views.

As will be described in detail below, the present disclosure is directed to a surgical robotic system, which includes a surgeon console, a control tower, and one or more mobile carts having a surgical robotic arm coupled to a setup arm. The surgeon console receives user input through one or more interface devices, which are processed by the control tower as movement commands for moving the surgical robotic arm and an instrument and/or camera coupled thereto. Thus, the surgeon console enables teleoperation of the surgical arms and attached instruments/camera. The surgical robotic arm includes a controller, which is configured to process the movement command and to generate a torque command for activating one or more actuators of the robotic arm, which would, in turn, move the robotic arm in response to the movement command.

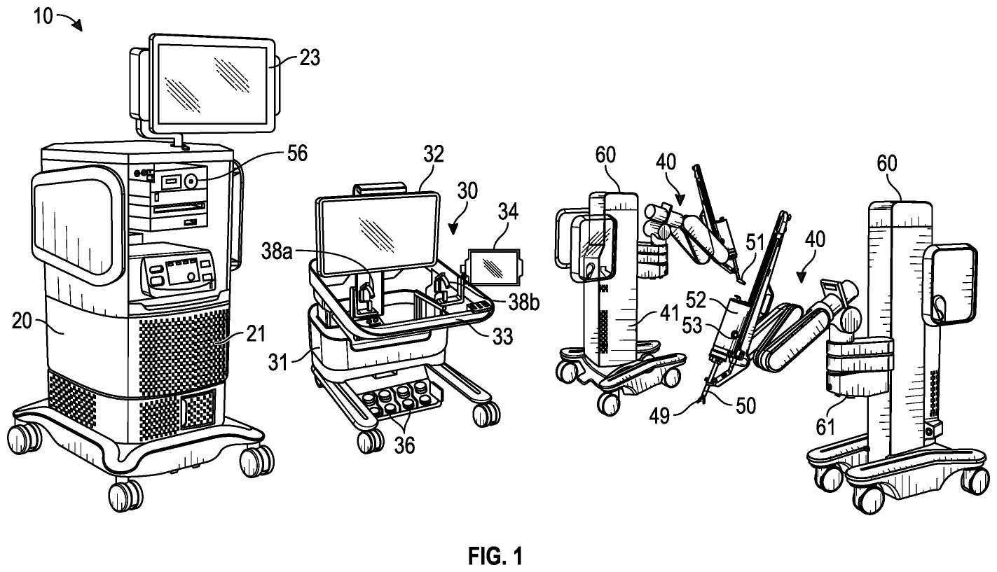

With reference to FIG. 1, a surgical robotic system 10 includes a control tower 20, which is connected to all of the components of the surgical robotic system 10 including a surgeon console 30 and one or more movable carts 60. Each of the movable carts 60 includes a robotic arm 40 having a surgical instrument 50 removably coupled thereto. The robotic arms 40 also couple to the movable cart 60. The robotic system 10 may include any number of movable carts 60 and/or robotic arms 40.

The surgical instrument 50 is configured for use during minimally invasive surgical procedures. In embodiments, the surgical instrument 50 may be configured for open surgical procedures. In embodiments, the surgical instrument 50 may be an endoscope, such as an endoscopic camera 51, configured to provide a video feed for the user. In further embodiments, the surgical instrument 50 may be an electrosurgical forceps configured to seal tissue by compressing tissue between jaw members and applying electrosurgical current thereto. In yet further embodiments, the surgical instrument 50 may be a surgical stapler including a pair of jaws configured to grasp and clamp tissue while deploying a plurality of tissue fasteners, e.g., staples, and cutting stapled tissue.

One of the robotic arms 40 may include the endoscopic camera 51 configured to capture video of the surgical site. The endoscopic camera 51 may be a stereoscopic endoscope configured to capture two side-by-side (i.e., left and right) images of the surgical site to produce a video stream of the surgical scene. The endoscopic camera 51 is coupled to a video processing device 56, which may be disposed within the control tower 20. The video processing device 56 may be any computing device as described below configured to receive the video feed from the endoscopic camera 51 and output the processed video stream.

The surgeon console 30 includes a first display 32, which displays a video feed of the surgical site provided by camera 51 of the surgical instrument 50 disposed on the robotic arm 40, and a second display 34, which displays a user interface for controlling the surgical robotic system 10. The first display 32 and second display 34 may be touchscreens allowing for displaying various graphical user inputs.

The surgeon console 30 also includes a plurality of user interface devices, such as foot pedals 36 and a pair of handle controllers 38a and 38b which are used by a user to remotely control robotic arms 40. The surgeon console further includes an armrest 33 used to support clinician's arms while operating the handle controllers 38a and 38b.

The control tower 20 includes a display 23, which may be a touchscreen, and outputs on the graphical user interfaces (GUIs). The control tower 20 also acts as an interface between the surgeon console 30 and one or more robotic arms 40. In particular, the control tower 20 is configured to control the robotic arms 40, such as to move the robotic arms 40 and the corresponding surgical instrument 50, based on a set of programmable instructions and/or input commands from the surgeon console 30, in such a way that robotic arms 40 and the surgical instrument 50 execute a desired movement sequence in response to input from the foot pedals 36 and the handle controllers 38a and 38b. The foot pedals 36 may be used to enable and lock the hand controllers 38a and 38b, repositioning camera movement and electrosurgical activation/deactivation. In particular, the foot pedals 36 may be used to perform a clutching action on the hand controllers 38a and 38b. Clutching is initiated by pressing one of the foot pedals 36, which disconnects (i.e., prevents movement inputs) the hand controllers 38a and/or 38b from the robotic arm 40 and corresponding instrument 50 or camera 51 attached thereto. This allows the user to reposition the hand controllers 38a and 38b without moving the robotic arm(s) 40 and the instrument 50 and/or camera 51. This is useful when reaching control boundaries of the surgical space.

Each of the control tower 20, the surgeon console 30, and the robotic arm 40 includes a respective computer 21, 31, 41. The computers 21, 31, 41 are interconnected to each other using any suitable communication network based on wired or wireless communication protocols. The term “network,” whether plural or singular, as used herein, denotes a data network, including, but not limited to, the Internet, Intranet, a wide area network, or a local area network, and without limitation as to the full scope of the definition of communication networks as encompassed by the present disclosure. Suitable protocols include, but are not limited to, transmission control protocol/internet protocol (TCP/IP), datagram protocol/internet protocol (UDP/IP), and/or datagram congestion control protocol (DCCP). Wireless communication may be achieved via one or more wireless configurations, e.g., radio frequency, optical, Wi-Fi, Bluetooth (an open wireless protocol for exchanging data over short distances, using short length radio waves, from fixed and mobile devices, creating personal area networks (PANs), ZigBee® (a specification for a suite of high level communication protocols using small, low-power digital radios based on the IEEE 122.15.4-1203 standard for wireless personal area networks (WPANs)).

The computers 21, 31, 41 may include any suitable processor (not shown) operably connected to a memory (not shown), which may include one or more of volatile, non-volatile, magnetic, optical, or electrical media, such as read-only memory (ROM), random access memory (RAM), electrically-erasable programmable ROM (EEPROM), non-volatile RAM (NVRAM), or flash memory. The processor may be any suitable processor (e.g., control circuit) adapted to perform the operations, calculations, and/or set of instructions described in the present disclosure including, but not limited to, a hardware processor, a field programmable gate array (FPGA), a digital signal processor (DSP), a central processing unit (CPU), a microprocessor, and combinations thereof. Those skilled in the art will appreciate that the processor may be substituted for by using any logic processor (e.g., control circuit) adapted to execute algorithms, calculations, and/or set of instructions described herein.

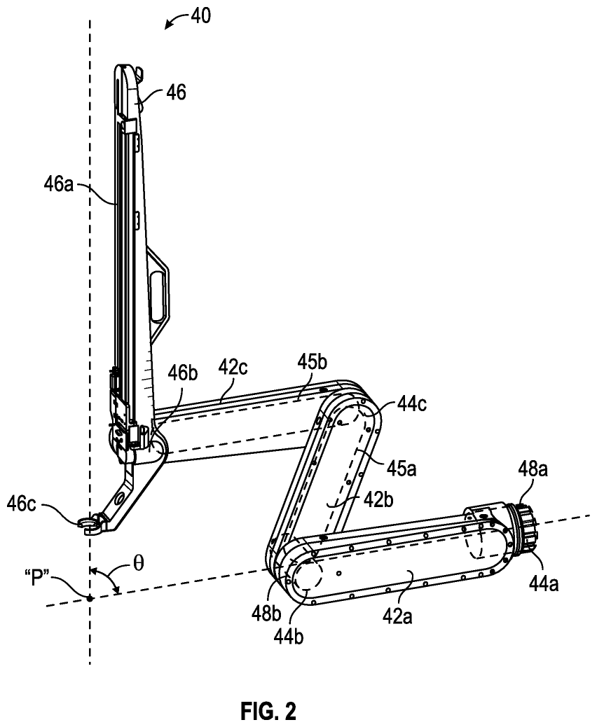

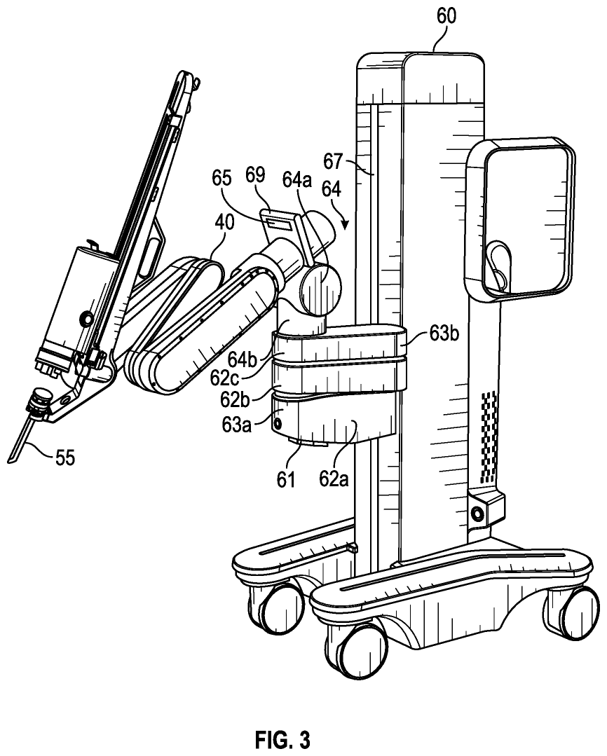

With reference to FIG. 2, each of the robotic arms 40 may include a plurality of links 42a, 42b, 42c, which are interconnected at joints 44a, 44b, 44c, respectively. Other configurations of links and joints may be utilized as known by those skilled in the art. The joint 44a is configured to secure the robotic arm 40 to the mobile cart 60 and defines a first longitudinal axis. With reference to FIG. 3, the mobile cart 60 includes a lift 67 and a setup arm 61, which provides a base for mounting of the robotic arm 40. The lift 67 allows for vertical movement of the setup arm 61. The mobile cart 60 also includes a display 69 for displaying information pertaining to the robotic arm 40. In embodiments, the robotic arm 40 may include any type and/or number of joints.

The setup arm 61 includes a first link 62a, a second link 62b, and a third link 62c, which provide for lateral maneuverability of the robotic arm 40. The links 62a, 62b, 62c are interconnected at joints 63a and 63b, each of which may include an actuator (not shown) for rotating the links 62b and 62b relative to each other and the link 62c. In particular, the links 62a, 62b, 62c are movable in their corresponding lateral planes that are parallel to each other, thereby allowing for extension of the robotic arm 40 relative to the patient (e.g., surgical table). In embodiments, the robotic arm 40 may be coupled to the surgical table (not shown). The setup arm 61 includes controls 65 for adjusting movement of the links 62a, 62b, 62c as well as the lift 67. In embodiments, the setup arm 61 may include any type and/or number of joints.

The third link 62c may include a rotatable base 64 having two degrees of freedom. In particular, the rotatable base 64 includes a first actuator 64a and a second actuator 64b. The first actuator 64a is rotatable about a first stationary arm axis which is perpendicular to a plane defined by the third link 62c and the second actuator 64b is rotatable about a second stationary arm axis which is transverse to the first stationary arm axis. The first and second actuators 64a and 64b allow for full three-dimensional orientation of the robotic arm 40.

The actuator 48b of the joint 44b is coupled to the joint 44c via the belt 45a, and the joint 44c is in turn coupled to the joint 46b via the belt 45b. Joint 44c may include a transfer case coupling the belts 45a and 45b, such that the actuator 48b is configured to rotate each of the links 42b, 42c and a holder 46 relative to each other. More specifically, links 42b, 42c, and the holder 46 are passively coupled to the actuator 48b which enforces rotation about a pivot point “P” which lies at an intersection of the first axis defined by the link 42a and the second axis defined by the holder 46. In other words, the pivot point “P” is a remote center of motion (RCM) for the robotic arm 40. Thus, the actuator 48b controls the angle θ between the first and second axes allowing for orientation of the surgical instrument 50. Due to the interlinking of the links 42a, 42b, 42c, and the holder 46 via the belts 45a and 45b, the angles between the links 42a, 42b, 42c, and the holder 46 are also adjusted in order to achieve the desired angle θ. In embodiments, some or all of the joints 44a, 44b, 44c may include an actuator to obviate the need for mechanical linkages.

The joints 44a and 44b include an actuator 48a and 48b configured to drive the joints 44a, 44b, 44c relative to each other through a series of belts 45a and 45b or other mechanical linkages such as a drive rod, a cable, or a lever and the like. In particular, the actuator 48a is configured to rotate the robotic arm 40 about a longitudinal axis defined by the link 42a.

With reference to FIG. 2, the holder 46 defines a second longitudinal axis and configured to receive an instrument drive unit (IDU) 52 (FIG. 1). The IDU 52 is configured to couple to an actuation mechanism of the surgical instrument 50 and the camera 51 and is configured to move (e.g., rotate) and actuate the instrument 50 and/or the camera 51. IDU 52 transfers actuation forces from its actuators to the surgical instrument 50 to actuate components an end effector 49 of the surgical instrument 50. The holder 46 includes a sliding mechanism 46a, which is configured to move the IDU 52 along the second longitudinal axis defined by the holder 46. The holder 46 also includes a joint 46b, which rotates the holder 46 relative to the link 42c. During endoscopic procedures, the instrument 50 may be inserted through an endoscopic access port 55 (FIG. 3) held by the holder 46. The holder 46 also includes a port latch 46c for securing the access port 55 to the holder 46 (FIG. 2).

The robotic arm 40 also includes a plurality of manual override buttons 53 (FIG. 1) disposed on the IDU 52 and the setup arm 61, which may be used in a manual mode. The user may press one or more of the buttons 53 to move the component associated with the button 53.

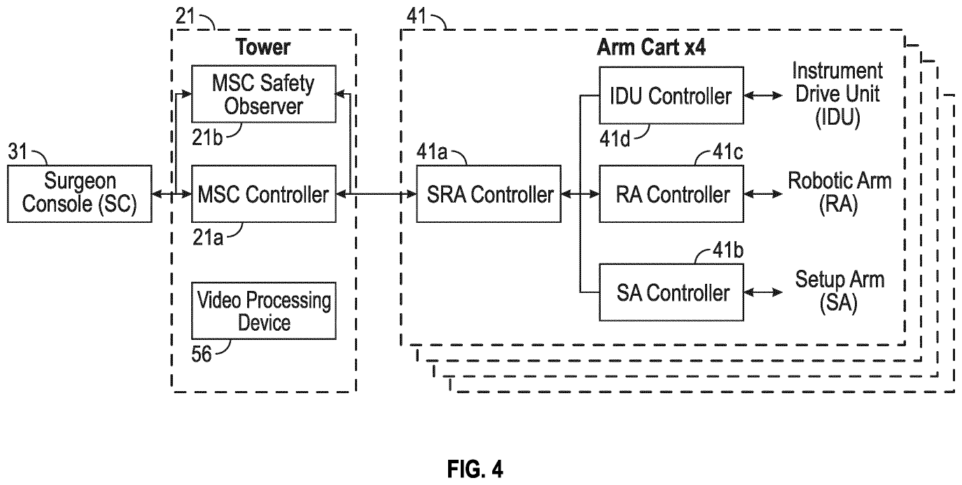

With reference to FIG. 4, each of the computers 21, 31, 41 of the surgical robotic system 10 may include a plurality of controllers, which may be embodied in hardware and/or software. The computer 21 of the control tower 20 includes a controller 21a and safety observer 21b. The controller 21a receives data from the computer 31 of the surgeon console 30 about the current position and/or orientation of the handle controllers 38a and 38b and the state of the foot pedals 36 and other buttons. The controller 21a processes these input positions to determine desired drive commands for each joint of the robotic arm 40 and/or the IDU 52 and communicates these to the computer 41 of the robotic arm 40. The controller 21a also receives the actual joint angles measured by encoders of the actuators 48a and 48b and uses this information to determine force feedback commands that are transmitted back to the computer 31 of the surgeon console 30 to provide haptic feedback through the handle controllers 38a and 38b. The safety observer 21b performs validity checks on the data going into and out of the controller 21a and notifies a system fault handler if errors in the data transmission are detected to place the computer 21 and/or the surgical robotic system 10 into a safe state.

The computer 41 includes a plurality of controllers, namely, a main cart controller 41a, a setup arm controller 41b, a robotic arm controller 41c, and an instrument drive unit (IDU) controller 41d. The main cart controller 41a receives and processes joint commands from the controller 21a of the computer 21 and communicates them to the setup arm controller 41b, the robotic arm controller 41c, and the IDU controller 41d. The main cart controller 41a also manages instrument exchanges and the overall state of the mobile cart 60, the robotic arm 40, and the IDU 52. The main cart controller 41a also communicates actual joint angles back to the controller 21a.

Each of joints 63a and 63b and the rotatable base 64 of the setup arm 61 are passive joints (i.e., no actuators are present therein) allowing for manual adjustment thereof by a user. The joints 63a and 63b and the rotatable base 64 include brakes that are disengaged by the user to configure the setup arm 61. The setup arm controller 41b monitors slippage of each of joints 63a and 63b and the rotatable base 64 of the setup arm 61, when brakes are engaged or can be freely moved by the operator when brakes are disengaged, but do not impact controls of other joints. The robotic arm controller 41c controls each joint 44a and 44b of the robotic arm 40 and calculates desired motor torques required for gravity compensation, friction compensation, and closed loop position control of the robotic arm 40. The robotic arm controller 41c calculates a movement command based on the calculated torque. The calculated motor commands are then communicated to one or more of the actuators 48a and 48b in the robotic arm 40. The actual joint positions are then transmitted by the actuators 48a and 48b back to the robotic arm controller 41c.

The IDU controller 41d receives desired joint angles for the surgical instrument 50, such as wrist and jaw angles, and computes desired currents for the motors in the IDU 52. The IDU controller 41d calculates actual angles based on the motor positions and transmits the actual angles back to the main cart controller 41a.

The robotic arm 40 is controlled in response to a pose of the handle controller controlling the robotic arm 40, e.g., the handle controller 38a, which is transformed into a desired pose of the robotic arm 40 through a hand eye transform function executed by the controller 21a. The hand eye function, as well as other functions described herein, is/are embodied in software executable by the controller 21a or any other suitable controller described herein. The pose of one of the handle controllers 38a may be embodied as a coordinate position and roll-pitch-yaw (RPY) orientation relative to a coordinate reference frame, which is fixed to the surgeon console 30. The desired pose of the instrument 50 is relative to a fixed frame on the robotic arm 40. The pose of the handle controller 38a is then scaled by a scaling function executed by the controller 21a. In embodiments, the coordinate position may be scaled down and the orientation may be scaled up by the scaling function. In addition, the controller 21a may also execute a clutching function, which disengages the handle controller 38a from the robotic arm 40. In particular, the controller 21a stops transmitting movement commands from the handle controller 38a to the robotic arm 40 if certain movement limits or other thresholds are exceeded and in essence acts like a virtual clutch mechanism, e.g., limits mechanical input from effecting mechanical output.

The desired pose of the robotic arm 40 is based on the pose of the handle controller 38a and is then passed by an inverse kinematics function executed by the controller 21a. The inverse kinematics function calculates angles for the joints 44a, 44b, 44c of the robotic arm 40 that achieve the scaled and adjusted pose input by the handle controller 38a. The calculated angles are then passed to the robotic arm controller 41c, which includes a joint axis controller having a proportional-derivative (PD) controller, the friction estimator module, the gravity compensator module, and a two-sided saturation block, which is configured to limit the commanded torque of the motors of the joints 44a, 44b, 44c.

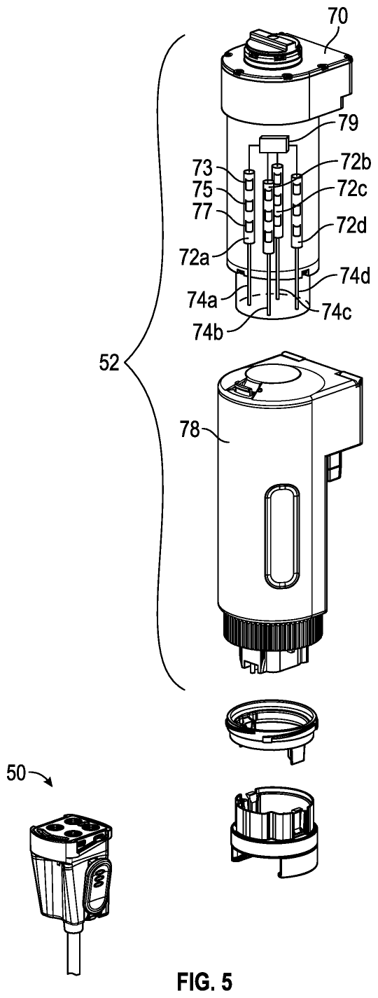

With reference to FIG. 5, the IDU 52 is shown in more detail and is configured to transfer power and actuation forces from its motors 72a, 72b, 72c. 72d to the instrument 50 to drive movement of components of the instrument 50, such as articulation, rotation, pitch, yaw, clamping, cutting, etc. The IDU 52 may also be configured for the activation or firing of an electrosurgical energy-based instrument or the like (e.g., cable drives, pulleys, friction wheels, rack and pinion arrangements, etc.).

The IDU 52 includes a motor pack 70 and a sterile barrier housing 78. Motor pack 70 includes motors 72a, 72b, 72c, 72d for controlling various operations of the instrument 50. The instrument 50 is removably couplable to IDU 52. As the motors 72a, 72b, 72c, 72d of the motor pack 70 are actuated, rotation of the drive transfer shafts 74a, 74b, 74c, 74d of the motors 72a, 72b, 72c. 72d, respectively, is transferred to the drive assemblies of the instrument 50. The instrument 50 is configured to transfer rotational forces/movement supplied by the IDU 52 (e.g., via the motors 72a, 72b, 72c, 72d of the motor pack 70) into longitudinal movement or translation of the cables or drive shafts to effect various functions of the end effector 49.

Each of the motors 72a, 72b, 72c, 72d includes a current sensor 73, a torque sensor 75, and an encoder sensor 77. For conciseness only operation of the motor 72a is described below. The sensors 73, 75, 77 monitor the performance of the motor 72a. The current sensor 73 is configured to measure the current draw of the motor 72a and the torque sensor 75 is configured to measure motor torque. The torque sensor 75 may be any force or strain sensor including one or more strain gauges configured to convert mechanical forces and/or strain into a sensor signal indicative of the torque output by the motor 72a. The encoder sensor 77 may be any device that provides a sensor signal indicative of the number of rotations of the motor 72a, such as a mechanical encoder or an optical encoder. Parameters which are measured and/or determined by the encoder sensor 77 may include speed, distance, revolutions per minute, position, and the like. The sensor signals from sensors 73, 75, 77 are transmitted to the IDU controller 41d, which then controls the motors 72a, 72b, 72c. 72d based on the sensor signals. In particular, the motors 72a, 72b, 72c, 72d are controlled by an actuator controller 79, which controls torque outputted and angular velocity of the motors 72a, 72b, 72c, 72d. In embodiments, additional position sensors may also be used, which include, but are not limited to, potentiometers coupled to movable components and configured to detect travel distances, Hall Effect sensors, accelerometers, and gyroscopes. In embodiments, a single controller can perform the functionality of the IDU controller 41d and the actuator controller 79.

The system 10 is configured to switch between a plurality of power modes automatically or manually. Power modes are defined by power and/or torque supplied by the motors 72a-d, which in turn, affects the grip strength of the end effector 49. There may be a plurality of power modes, including a full power mode at which the motors 72a-d are powered at a preset (e.g., 100%), one or more high power modes (e.g., above 100%), and one or more low power modes (e.g., below 100%).

Automatic adaptive mode switching may be performed by the by any suitable controller of the system 10, e.g., IDU controller 41d, controller 21a, etc. For simplicity, reference is made to controller 21a, which is configured to execute algorithms, which are embodied as software instructions, for processing various events and operating in an adaptive manner to switch between power modes.

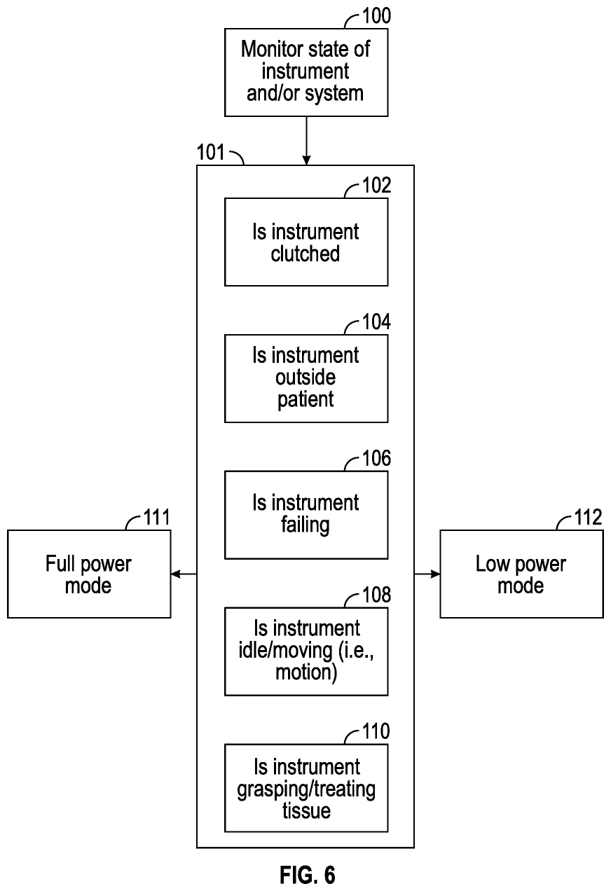

With reference to FIG. 6, a method for automatic/adaptive switching between a plurality of power modes continuous monitoring of various system and instrument parameters at step 100. Monitoring may include an event tracker configured to track various parameters of the system 10, including operation time, phase identification (e.g., access port setup, cutting, extraction, stitching, etc.). Furthermore, user inputs from the surgeon console 30 and other user input interfaces are also monitored, which includes receiving sensor data from sensors 73, 75, 77 and determining various parameters of the instrument 50 and the IDU 52, such as yaw, pitch, jaw angles, insertion depth, grasping force, current draw, etc.

A surgical procedure can include multiple phases, and each phase can include one or more surgical actions. A “surgical action” can include an incision, a compression, a stapling, a clipping, a suturing, a cauterization, a sealing, or any other such actions performed to complete a phase in the surgical procedure. A “phase” represents a surgical event that is composed of a series of steps (e.g., closure). A “step” refers to the completion of a named surgical objective (e.g., hemostasis).

In some embodiments, a machine learning processing system may be used to detect phases and may include a phase detector that uses the machine learning models to identify a phase within the surgical procedure (“procedure”). Phase detector uses a particular procedural tracking data structure from a list of procedural tracking data structures. Phase detector selects the procedural tracking data structure based on the type of surgical procedure that is being performed. In one or more examples, the type of surgical procedure is predetermined or input by the user. The procedural tracking data structure identifies a set of potential phases that can correspond to a part of the specific type of procedure.

In some examples, the procedural tracking data structure can be a graph that includes a set of nodes and a set of edges, with each node corresponding to a potential phase. The edges can provide directional connections between nodes that indicate (via the direction) an expected order during which the phases will be encountered throughout an iteration of the procedure. The procedural tracking data structure may include one or more branching nodes that feed to multiple next nodes and/or can include one or more points of divergence and/or convergence between the nodes. In some instances, a phase indicates a procedural action (e.g., surgical action) that is being performed or has been performed and/or indicates a combination of actions that have been performed. In some instances, a phase relates to a biological state of a patient undergoing a surgical procedure. For example, the biological state can indicate a complication (e.g., blood clots, clogged arteries/veins, etc.), pre-condition (e.g., lesions, polyps, etc.). In some examples, the machine learning models are trained to detect an “abnormal condition,” such as hemorrhaging, arrhythmias, blood vessel abnormality, etc.

Each node within the procedural tracking data structure can identify one or more characteristics of the phase corresponding to that node. The characteristics can include visual characteristics. In some instances, the node identifies one or more tools that are typically in use or availed for use (e.g., on a tool tray) during the phase. The node also identifies one or more roles of people who are typically performing a surgical task, a typical type of movement (e.g., of a hand or tool), etc. Thus, phase detector can use the segmented data generated by machine learning execution system that indicates the presence and/or characteristics of particular objects within a field of view to identify an estimated node to which the real image data corresponds. Identification of the node (i.e., phase) can further be based upon previously detected phases for a given procedural iteration and/or other detected input (e.g., verbal audio data that includes person-to-person requests or comments, explicit identifications of a current or past phase, information requests, etc.).

The phase detector outputs the phase prediction associated with a portion of the video data that is analyzed by the machine learning processing system. The phase prediction is associated with the portion of the video data by identifying a start time and an end time of the portion of the video that is analyzed by the machine learning execution system. The phase prediction that is output can include an identity of a surgical phase as detected by the phase detector based on the output of the machine learning execution system. Further, the phase prediction, in one or more examples, can include identities of the structures (e.g., instrument, anatomy, etc.) that are identified by the machine learning execution system in the portion of the video that is analyzed. The phase prediction can also include a confidence score of the prediction. Other examples can include various other types of information in the phase prediction that is output.

The controller 21a is configured to automatically switch the operating mode for the IDU 52 between a full power or high power mode at step 111 and one or more low power modes at step 112 based on one or more factors. Each of the IDUs 52 operates in a default, or previously selected mode until one or more of the conditions are detected by the controller 21a. In embodiments, the default mode may be the full power mode. The conditions could be one or more factors or user actions.

At step 101, the controller 21a determines whether a one of the monitored parameters or user actions have been detected and if so, the controller 21a switches between the power modes based on the parameter and preprogrammed power mode setting. Step 101 includes monitoring for specific events as listed below in steps 102-110.

At step 102, the controller 21a determines whether the user performing a clutching input. When instrument 50 is clutched, the controller 21a enters the IDU 52 into a low power mode, during which torque on all four motors 72a-d may be reduced equally to reduce tension on cables of the instrument 50 without moving the end effector 49. After the instrument 50 is clutched back, i.e., control is resumed, the controller 21a restores the motors 72a-d to the full power mode. Before switching to the lower power mode, the controller 21a is configured to record current torque applied during the full power mode. After clutching back, the torque applied by the motors 72a-d is then restored to the recorded torque after switching back to the full power mode from the low power mode.

In embodiments, the surgeon console 30 may also track engagement of the surgeon with the surgeon console 30 by monitoring head position and/or gaze of the user. The controller 21a may select a low power mode in response to the surgeon console 30 detecting that the surgeon is disengaged. The controller 21a may select the full power mode once the surgeon is engaged.

At step 104, the controller 21a determines whether the instrument 50 is positioned inside or outside the patient. Location of the instrument 50 may be confirmed using positional feedback of the IDU 52 on the sliding mechanism 46a. In embodiments, location may also be determined by using computer vision, i.e., analyzing the feed from the camera 51 or following calibration of the instrument 50, which is performed inside a patient. If the instrument 50 is outside the patient, the controller 21a switches the IDU 52 to the lower power mode, during which torque on all four motors 72a-d may be reduced equally to reduce tension on cables of the instrument 50 without moving the end effector 49. Once the instrument 50 is inserted into the patient, the controller 21a switches the IDU 52 to the full power mode. Thus, as the instrument 50 is being retracted, the controller 21a enters the low power mode.

At step 106, the controller 21a monitors life of the instrument 50 and enters the lower power mode based on the remaining life of the instrument 50. Instrument life may be tracked during the procedure based on time and/or type of use of the instrument 50. Time may be tracked starting from the initial use of the instrument 50 (i.e., coupling and actuation by the IDU 52) and use may be tracked based on number, power, duration of activations of the instrument 50 and/or the IDU 52. Upon expiration of the instrument life, the controller 21a switches the IDU 52 into the low power mode from the default mode, e.g., full power mode.

The controller 21a is also configured to monitor breakage of the instrument 50 and/or IDU 52. The controller 21a receives sensor data from the sensors 73, 75, 77 and compares the data to specific thresholds that are indicative of mechanical failure, e.g., cable snap or fray. In embodiments, machine learning may be used to automatically detect when the instrument 50 is at risk of failing. If the instrument 50 is about to fail, the instrument 50 enters into a low power mode for the duration of use of the instrument 50.

In further embodiments, breakage may also be detected using computer vision by analyzing the feed from the camera 51, e.g., limping end effector 49. Once mechanical failure is detected, the controller 21a disables one or more of the motors 72a-d responsible for actuating the broken cable and activates the remaining motors 72a-d in a low power mode to maintain the end effector 49 in a stationary position, enabling retraction of the instrument 50. The instrument 50 may be retracted, while maintaining low power mode.

At step 108, the controller 21a monitors whether the instrument 50 is idle. The instrument 50 may be idle for a set period of time, i.e., a spare instrument that is not currently being used, before a low power mode is activated. The controller 21a maintains a timer to determine idle time and at the expiration of the timer, the IDU 52 enters a low power mode. When motion is commanded, namely, instrument 50 is selected and the handle controller 38a/38b is moved, the system 10 instructs the IDU 52 to restore to full power mode. In embodiments, data from the sensors 73, 75, 77 and/or computer vision may be used to detect when the instrument 50 is idle. When the end effector 49 is closed but not grasping anything, the system 10 may also enter the low power mode.

Similar to clutching, the controller 21a may record the current torque before entering the low power mode, then reduce torque on all four motors 72a-d equally to reduce tension on cables of the instrument 50 at idle mode. After exiting the lower power mode, the controller 21a restores the recorded torque on all four motors 72a-d and then restart teleoperation.

During step 108, the controller 21a also monitors rate of acceleration on user controls inputs, i.e., hand controllers 38a and 38b. The controller 21a is configured to compare the rate of acceleration to a threshold for acceleration rate of motion to determine if the instrument 50 is being moved/relocated and enters the low power mode. Once instrument 50 is stationary, the controller 21a enters the IDU 52 into the full power mode to ensure the end effector 49 can fully grasp/treat tissue. In embodiments, the controller 21a also monitors hand tremor at hand controllers 38a and 38b and compensates for any resulting movement. The controller 21a may use thresholds for filtering motion to avoid needlessly switching between low and full power modes.

During step 110, the controller 21a monitors status of the end effector 49 and adjusts the power mode accordingly. The controller 21a determines whether the end effector 49 is about to grasp tissue based on user input commands, e.g., opening and commencing jaw closure, and/or torque monitoring to determine tissue contact with the end effector 49. Upon detection of grasping, the controller 21a is configured to switch the IDU 52 to the full power mode to ensure that tissue is grasped securely. In embodiments, grasping force may be adjusted based on the type of instrument or procedure being performed since grasping force varies for different instruments. For instance, when using a bipolar vessel sealer, grasping force may be reduced, since tissue is compressed at a specific force threshold, thus the lower power mode may be enabled by the controller 21a. Conversely, when shears are used, over-grasping is enabled, thus full power mode is enabled to ensure tissue is completely severed. The controller 21a is configured to dynamically switch between full and low power modes based on the function and grasping state of the end effector 49.

In embodiments, the controller 21a is also configured to determine the phase of the surgical procedure (e.g., insertion, retraction, stapling, stitching, etc.) and to switch between low and full power modes for various components of the system 10, i.e., IDUs 52. Surgical procedures are organized as a series of steps, which may be loaded into the system 10. Power mode settings for specific devices may then be selected for specific instruments 50 based on the steps of the procedure. In embodiments, machine learning may also be used to analyze the timing, movements, and activities of the robotic arms 40 during surgical procedures, to predict the next surgical step and the associated power mode for the instruments 50.

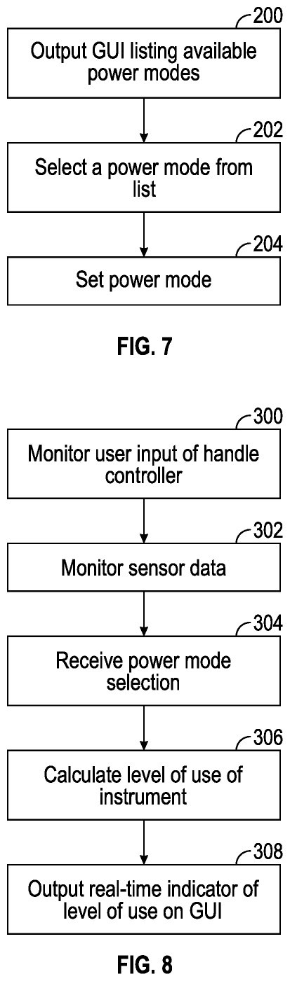

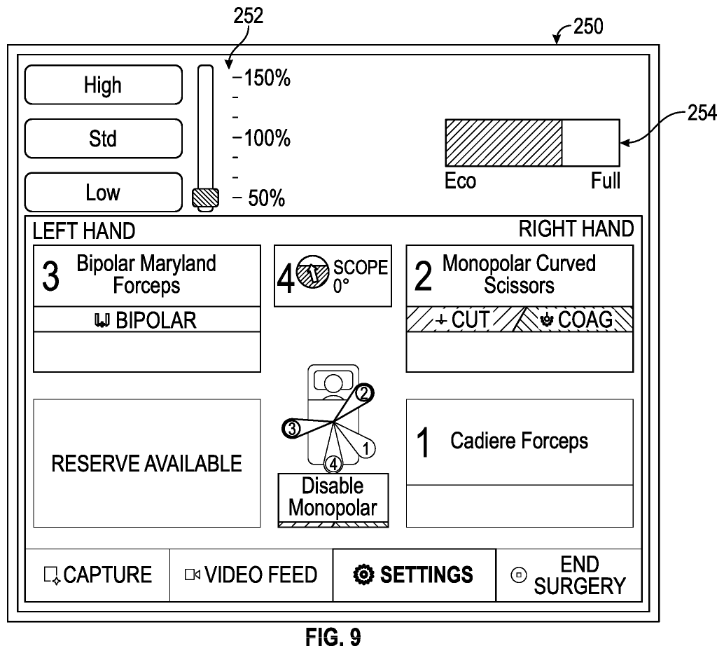

With reference to FIG. 7, a method for user-selected switching between power modes, i.e., a full power mode and one or more additional low power modes, includes outputting a mode selection graphical user interface (GUI) 250 (FIG. 9) at step 200. The GUI 250 may be touch-enabled and may include one or more windows, menus, icons, tabs, a slider, text boxes (e.g., to enter percentage) or any other suitable selection interface 252. The GUI 250 may be displayed on one of the displays 23, 32, 34 listing a plurality of power levels, allowing the surgeon to select the power level at step 202. In particular, the surgeon may select a power mode for each instrument type that is being used and/or each specific instrument 50. Power mode is related to the grasping force since the power supplied to the motors 72a-d is related to the torque output, which in turn, is related to the grasping force.

In addition to full and low power modes, there may be a plurality of modes corresponding to different grasping forces. In embodiments, a low power mode may also be categorized in the GUI 250 as a “gentle” mode for handling thin, fragile, or critical tissue, a standard power mode, and a secure power mode for handling thick or difficult-to-manipulate tissue. These modes may be selected by the surgeon or automatically triggered based on a surgical phase. In embodiments, the power mode may be adjusted based on tissue slippage. Thus, grasping may commence at a low power mode, and upon detecting slippage of tissue, a higher power mode is engaged to increase the grasping force.

At step 204, the selected power mode is set for the IDU 52 and is in place until a new power mode is selected as described above in steps 200 and 202. In embodiments, a default low level may be set by the system 10 to maximize the life of the instrument 50 and allow for subsequent increases of the grasping force by selecting a desired power mode. The selectable power mode may be adjusted, i.e., limited, based on the remaining life of the instrument. Torque, use time, and other parameters are also tracked during use of the instrument 50, and these parameters may be used to limit the selected power mode.

In further embodiments, power mode may be selected based on a grip force imparted on the handle controllers 38a and 38b, i.e., based on a level of force the surgeon is grasping the handle or paddles of the handle controllers 38a and 38b. The handle controllers 38a and 38b include one or more sensors (e.g., strain gauges disposed in the handles) configured to measure grasping force imparted by the surgeon. The system 10 then selects a grasping level of the instrument 50 based on the grasping force imparted on the handle controllers 38a and 38b. In embodiments, the handle controllers 38a and 38b may include a button that when engaged, increases the grasping force by a preset amount above the currently-selected grasping force.

The surgeon console 30 may also be configured to receive user settings, including specific power modes based on user preferences. User settings may be stored in a database on the surgeon console 30, retrieved from a remote database, and/or loaded from a storage device, e.g., memory card, associated with the surgeon. The user settings may include power mode selections for each instrument type and/or procedure. Thus, as the surgeon commences the procedure, the default power mode settings are loaded from the user settings by the surgeon console 30. The surgeon may override the default power mode using the GUI 250 or other selection steps described above.

FIG. 8 shows a method for displaying real-time life and/or level of use of the instrument 50 based on the selected power mode, type of use of the instrument 50, and other sensor data from the instrument 50. At step 300, the user inputs of the handle controllers 38a and 38b are monitored by the controller 21a, which includes measuring velocity and acceleration of movement of the handle controllers 38a and 38b. This data is used to determine how delicate or harsh the instrument 50 is being used, since the type of use affects the life of the instrument, i.e., delicate use decreases life of the instrument 50 at a slower rate whereas harsh use decreases life of the instrument 50 at a faster rate.

At step 302, data from the sensors 73, 75, 77 is also monitored by the controller 21a to determine level of use of the instrument 50. At step 304, the currently selected power mode is also provided to the controller 21a. At step 306, the controller 21a determines current level of use of the instrument 50 based on the user inputs, selected power level, and/or sensor data.

At step 308, a real-time indicator 254 of instrument use is displayed on the GUI 250 shown on one or more the displays 23, 32, 34 (FIG. 9). The indicator may be an alphabetical or numerical indicator, e.g., percentage, a color indicator, a hysteresis gauge or bar, combinations thereof, etc. The indicator provides real-time information to the user regarding current use impacting remaining usage life of the instrument 50. In embodiments, the indicator may be a running average indicator with any suitable time window (e.g., 1 minute to 10 minutes). The indicator provides useful feedback to the user encouraging gentler use of the instrument 50 to maximize remaining life.

It will be understood that various modifications may be made to the embodiments disclosed herein. Therefore, the above description should not be construed as limiting, but merely as exemplifications of various embodiments. Those skilled in the art will envision other modifications within the scope and spirit of the claims appended thereto.

What is claimed is:

1. A surgical robotic system comprising:
a robotic arm including an instrument and an instrument drive unit configured to couple to and to actuate the instrument;
a surgeon console including a handle controller configured to control the robotic arm and the instrument; and
a processor configured to:
monitor a parameter of the instrument;
select a power mode from a plurality of power modes for the instrument drive unit based on the parameter, the plurality of power modes includes a full power mode during which the instrument drive unit is fully powered and a low power mode during which the instrument drive unit is partially powered; and
set the instrument drive unit to the selected power mode.

2. The surgical robotic system according to claim 1, wherein the processor is further configured to determine whether the handle controller is clutched and to select the power mode from the plurality of power modes based on a clutch status.

3. The surgical robotic system according to claim 2, wherein the processor is further configured to select the low power mode when the handle controller is clutched out.

4. The surgical robotic system according to claim 2, wherein the processor is further configured to select the full power mode when the handle controller is clutched in.

5. The surgical robotic system according to claim 1, wherein the processor is further configured to determine whether the instrument is inside or outside a patient.

6. The surgical robotic system according to claim 5, wherein the processor is further configured to select the low power mode when the instrument is disposed outside the patient.

7. The surgical robotic system according to claim 6, wherein the processor is further configured to select the full power mode when the instrument is disposed inside the patient.

8. A surgical robotic system comprising:
a robotic arm including an instrument and an instrument drive unit configured to couple to and to actuate the instrument; and
a processor configured to:
monitor a parameter of the instrument;
select a power mode from a plurality of power modes for the instrument drive unit based on the parameter, the plurality of power modes includes a full power mode during which the instrument drive unit is fully powered and a low power mode during which the instrument drive unit is partially powered; and
set the instrument drive unit to the selected power mode.

9. The surgical robotic system according to claim 8, wherein the processor is further configured to determine whether the instrument is inside or outside a patient.

10. The surgical robotic system according to claim 9, wherein the processor is further configured to select the low power mode when the instrument is disposed outside the patient.

11. The surgical robotic system according to claim 9, wherein the processor is further configured to select the full power mode when the instrument is disposed inside the patient.

12. The surgical robotic system according to claim 8, wherein the processor is further configured to determine whether the instrument is idle or is in use.

13. The surgical robotic system according to claim 12, wherein the processor is further configured to select the low power mode when the instrument is idle.

14. The surgical robotic system according to claim 12, wherein the processor is further configured to select the full power mode when the instrument is in use.

15. The surgical robotic system according to claim 8, wherein the processor is further configured to determine whether the instrument is grasping tissue.

16. The surgical robotic system according to claim 15, wherein the processor is further configured to select the low power mode when the instrument is not grasping tissue.

17. The surgical robotic system according to claim 15, wherein the processor is further configured to select the full power mode when the instrument is grasping tissue.

18. A method for controlling a surgical robotic system, the method comprising:
monitoring a parameter of an instrument coupled to an instrument drive unit disposed on a robotic arm, the instrument drive unit configured to couple to and to actuate the instrument;
monitoring input at a surgeon console including a handle controller configured to control the robotic arm and the instrument;
selecting a power mode from a plurality of power modes for the instrument drive unit based on the parameter, the plurality of power modes includes a full power mode during which the instrument drive unit is fully powered and a low power mode during which the instrument drive unit is partially powered; and
setting the instrument drive unit to the selected power mode.

19. The method according to claim 18, further comprising:
determining at a processor whether the instrument is idle or is in use;
selecting the low power mode when the instrument is idle; and
selecting the full power mode when the instrument is in use.

20. The method according to claim 18, further comprising:
determining at a processor whether the instrument is grasping tissue;
selecting the low power mode when the instrument is not grasping tissue; and
selecting the full power mode when the instrument is grasping tissue.
